(19) United States
(12) Patent Application Publication
(10) Pub. No.: US 2024/0363202 A1
(43) Pub. Date: Oct. 31, 2024

(54) SYSTEM AND METHOD FOR EMERGENCY FATE AND TRANSPORT ANALYSIS

(71) Applicant: True Elements, Inc., Naples, FL (US)

(72) Inventors: William C. LOUISELL, III, Mt. Pleasant, SC (US); David BANKSTON, Naples, FL (US)

(21) Appl. No.: 18/645,105

(22) Filed: Apr. 24, 2024

Related U.S. Application Data
(60) Provisional application No. 63/462,451, filed on Apr. 27, 2023.

(51) Int. Cl. G16C 20/30; G16C 20/70

(52) U.S. Cl. CPC G16C 20/30; G16C 20/70

(57) ABSTRACT

Systems, methods, and non-transitory computer-readable media for fate and transport analysis, and more specifically to determining how contaminants change as they move through the environment. Systems can receive chemical contamination data associated with a geographic area, then identify, by executing at least one chemical detection machine learning model using the chemical contamination data, chemical contaminants within the geographic area. The systems can then predict, by executing at least one chemical dispersion machine learning model using at the chemical contaminants, a chemical-specific dispersion of the chemical contaminants within the geographic area. Based on that chemical-specific dispersion, the system can generate at least one assessment.

### CROSS-REFERENCE

This application claims priority to U.S. provisional patent application No. 63/462,451, filed Apr. 27, 2023, the contents of which are incorporated herein in their entirety.

### BACKGROUND

### 1. Technical Field

The present disclosure relates to fate and transport analysis, and more specifically to determining how contaminants change as they move through the environment.

### 2. Introduction

“Fate and transport” models estimate the movement and chemical alteration of contaminants as they move through air, soil, or water. However, in emergency situations identifying what the chemical contaminants are, where those different chemicals may be spreading, the rates at which the chemicals are spreading, and who should be contacted when the levels of contaminants represent a health risk, are all problems that current fate and transport models cannot solve.

### SUMMARY

Additional features and advantages of the disclosure will be set forth in the description that follows, and in part will be understood from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims, or can be learned by the practice of the principles set forth herein.

Disclosed are systems, methods, and non-transitory computer-readable storage media which provide a technical solution to the technical problem described. A method for performing the concepts disclosed herein can include: receiving, at a computer system, chemical contamination data associated with a geographic area; identifying, via at least one processor of the computer system executing at least one chemical detection machine learning model using the chemical contamination data, chemical contaminants within the geographic area; predicting, via the at least one processor executing at least one chemical dispersion machine learning model using at the chemical contaminants, a chemical-specific dispersion of the chemical contaminants within the geographic area; and generating, from the computer system, at least one assessment based on the chemical-specific dispersion.

A system configured to perform the concepts disclosed herein can include: receiving chemical contamination data associated with a geographic area; identifying, by executing at least one chemical detection machine learning model using the chemical contamination data, chemical contaminants within the geographic area; predicting, by executing at least one chemical dispersion machine learning model using at the chemical contaminants, a chemical-specific dispersion of the chemical contaminants within the geographic area; and generating at least one assessment based on the chemical-specific dispersion.

A non-transitory computer-readable storage medium configured as disclosed herein can have instructions stored which, when executed by at least one processor, cause the at least one processor to perform operations which include: receiving chemical contamination data associated with a geographic area; identifying, by executing at least one chemical detection machine learning model using the chemical contamination data, chemical contaminants within the geographic area; predicting, by executing at least one chemical dispersion machine learning model using at the chemical contaminants, a chemical-specific dispersion of the chemical contaminants within the geographic area; and generating at least one assessment based on the chemical-specific dispersion.

### BRIEF DESCRIPTION OF THE DRAWINGS

FIG. 1 illustrates an example system embodiment;

FIG. 2 illustrates an example of contaminants dispersing over time;

FIG. 3 illustrates an example of normal monitoring of permitted discharges;

FIG. 4 illustrates an example of a multilayer situational awareness map;

FIG. 5 illustrates an example method embodiment; and

FIG. 6 illustrates an example computer system.

### DETAILED DESCRIPTION

Various embodiments of the disclosure are described in detail below. While specific implementations are described, this is done for illustration purposes only. Other components and configurations may be used without parting from the spirit and scope of the disclosure.

Despite efforts at safety and regulation, chemical spills are a regular occurrence. When those chemical spills occur, the resulting chemicals can escape into the atmosphere, into water sources, and into the surrounding land, resulting in contamination that may be dangerous for inhabitants, agriculture, livestock, and/or wildlife. In such instances, quickly discovering what chemicals were released, how dangerous those chemicals are, where those chemicals are moving (and at what concentrations), and warning various parties that may be at risk (or providing other notifications regarding assessments to parties), are all necessary steps to preventing the chemical spill from becoming an ecological disaster.

To that end, systems configured as disclosed herein can perform steps including: (1) Discovering what chemicals have been released into an environment; (2) Determining where the chemicals will be moving, and at what concentrations; and (3) Issuing warnings to one or more entities that may be affected by the chemicals.

Regarding discovery of the chemicals spilled, in a perfect world the shipping entity (e.g., the train, the semi-truck, the pipeline, the barge, the cargo ship, etc.) or industrial activity responsible for the spill would have an accurate accounting of the chemicals which were being transported or discharged, and would release that information either publicly or to the system configured as disclosed herein. However, often such entities either do not have accurate lists of the chemicals being shipped, or are reticent to provide the lists publicly.

Because of the difficulty in obtaining the list of spilled chemicals directly from the shipping and/or industrial entity, systems configured as disclosed herein can scrape data from social media and/or news websites (e.g., FACEBOOK, INSTAGRAM, TWITTER, TIK-TOK, etc.), where users and/or reporters can post experiences, symptoms, and content, along with time/location data for where the symptoms/experiences occurred. The system can collect/scrape the data using APIs (Application Programming Interfaces), aggregate similar data together, and review the aggregated data that is being posted in a geographic region around a spill for keywords associated with the chemicals and/or symptoms. For example, if a spill occurred in location “X”, and multiple users posted to TWITTER that they live within a certain distance from X, and that they smell something odd with a burning sensation, or an acrid burning odor, the system can analyze that aggregated data, capture the adjectives used to describe what is being smelled, and combine it with other known information. This scraping and aggregating of data can also occur even when no known spill or other contamination is known, allowing the system to use such data to identify instances of contamination when nothing has been officially reported within formal media channels. In other words, systems configured as described herein can constantly be scraping social media, forums, and/or news websites, allowing the system to constantly be determining if an unreported spill has occurred based on scraped data.

The data used to discover chemical contaminants can also include sensor data. As sensors collect data, that information can be relayed to a computing system (e.g., one or more servers) which compare the sensor data to known behaviors of potential contaminants. For example, if a contaminant is known to alter the pH of a body of water, and a pH sensor located in a body of water detects a change, that change in pH can be reported to the system and the system can identify the potential set of contaminants in the body of water.

The determination process can be guided by an AI (Artificial Intelligence) engine. The AI engine can identify a location of interest and be tuned to potential sources within the watershed (e.g., USGS (United States Geological Survey) Hydrologic Unit at the 12-digit level and the specific Stream Reach National Hydrography Dataset identification number) of first indication. The AI engine can then employ an upstream search algorithm to identify all upstream reaches and reservoirs extending to the headwaters of the watershed (e.g., USGS Hydrologic Unit at the 4-digit level). With source mapping trace-lines identified, the AI engine can poll data systems for EPA (Environmental Protection Agency)-permitted activities and compile a list of EPA-regulated contaminants, their EPA-reported direct surface water discharge quantities and indirect transfers to wastewater treatment systems, and locations of permitted discharge points via live, API-driven reference to the EPA-managed Discharge Monitoring Reporting (DMR) system and/or the EPA-managed Toxic Release Inventory (TRI) system, (and/or similar systems managed at the state, and/or local level) along with publicly viewable live sensor data for the various sensors associated with Federal, Regional, State, Local, and Hyper-Local water quality monitoring programs to collect initial data for analysis the AI Engine to identify the likely chemistry of the contaminants.

With the likely list and permitted quantities of the contaminants, the AI engine can proceed to create a range of accidental discharge possibilities (shipping, manufacturing, and/or storage spills) such that a preliminary estimate of residual levels is generated for each source point for the likely contaminant(s) of influence. This process can narrow the list by identifying those sources and contaminants that are at a sufficient distance and/or on non-related up/down stream reaches that the potential for ‘spike’ level arrivals at the location of interest are deemed less likely. The methods and systems disclosed herein can provide early warning, reducing the potential of an accidental or non-permit compliant release of chemicals in excess of normally expected levels. To that end, the AI engine can examine all possible source-contaminant pairs and place them in a multi-tier source list in which source point-contaminant pairs are binned into categories/bins (such as, but not limited to: ‘most likely’, ‘likely’, ‘less likely’, and ‘least likely’).

With the potential source point-contaminant pairs binned, the AI engine can turn from the Identification Task to the Tracking Task. Within the Tracking Task, the first step is for the AI engine to enlist the available public and subscribed private water quality and physical property sensors located along the likely paths over which the contaminants will travel. These sensors are digitally grouped into a ‘family’ of sensors, such that disparate information from the applicable population sensors can be assessed across multiple measures that can provide clarifying information. As an example, if three sensors are located along a series of reaches and Sensor A detects a pH spike as compared with historical readings for that particular sensor, Sensor B detects a Biological Oxygen Demand (BOD) change as compared with historical readings for that particular sensor, and Sensor C detects a significant deviation in turbidity as compared with historical readings for that particular sensor, then the AI engine can reference a library of past events and their ‘markers’ such that a comparison for similarity can be made and a similarity score can be generated. The AI engine can also document the sensor data and/or the comparison, providing future ability to compare the ‘markers’.

With the path mapped, the AI engine can turn to the task of prediction. Specifically, based on the aggregated social media scraped data, environmental reporting data, and the sensor data, and/or any “official” data (e.g., manifest lists, time/location of a spill, etc.), the system can make predictions regarding (1) what contaminants have been released, (2) the concentrations of those contaminants, and (3) expected times of arrivals for points of interest downstream. Preferably, the system uses one or more neural network models or other form of AI (Artificial Intelligence) to make such a prediction. For example, a neural network model can be trained using a list of known contaminants from a previous spill and a list of words people used on social media to describe their experiences (along with synonyms and the like) with that previous spill. When the model is trained and executed, the resulting code receive as inputs words of people to describe their current circumstances regarding a new/current spill and, if similar to words used by previous users, a similar chemical can be predicted to be present at the current spill. Likewise, a neural network model can be trained using a list of known contaminants from a previous spill and sensor data from that previous spill. When the neural network model is trained, the resulting code can take as an input the sensor data and output a list of one or more potentially present chemicals. In some configurations, a neural network can be trained to use one or more of scraped data, environmental reporting data, sensor data, and/or “official” data (e.g., released manifests) to predict the chemicals present.

Once the types of chemicals present have been discovered, the system can reference information in the various Environmental Protection Agency (EPA) sources such as the Toxic Release Inventory system (or a similar system) to identify a likely chemistry of the contaminants, which can be used to determine future behaviors of the chemicals and any possible danger associated with the chemicals.

With the predicted chemicals and their associated chemistry, the system can predict what amounts and/or concentrations of chemicals will be found within a system (e.g., a riverine system, an atmospheric system, a soil system) over time. That is, rather than calculate only how much of the chemical will remain at the spill location over time, the system can determine how much of the chemical will be present in the atmosphere, downriver, and/or in the soil within the geographic region surrounding the spill location.

To do so, the system first identifies, for each chemical detected based on their chemistry, a chemistry specific dissipation curve. The dissipation curve identifies to what degree the chemical will transform into another form (i.e., dissipation), or otherwise disperse (or persist) in an environment (e.g., water, soil, and/or air) over time. Some non-limiting examples of how dissipation curves show chemicals disperse include: linearly (e.g., the amount or concentration decreases at the same rate every hour), in a step fashion (e.g., the amount or concentration stays steady for a period, followed by a sharp reduction), exponentially (e.g., the amount or concentration falls off faster over time as the chemical travels, is exposed to other elements, etc.).

Once the dissipation curves for each predicted chemical is selected, the system can use atmospheric, soil, and/or hydrological models to predict how each chemical will spread over time. Such models predict how water and/or air moves throughout the geographic area, and (when combined with the dissipation curve data) can predict how much of a chemical will transfer from location A to location B in a time period. The models can also predict how much of the contaminants present in the air, water, and/or soil will transfer between those respective zones (i.e., from air to water or vice versa, from water to soil or vice versa, etc.). These predictions can occur for all locations within a geographic region associated with the spill site. That geographic region can include, for example, the area where the wind will be blowing, the downstream locations (both from where the spill occurred and where chemicals may be deposited from the air), etc. To that end, the atmospheric, soil, and/or hydrological models can use inputs including (but not limited to) weather forecasts, the chemical-specific dissipation curves, current water levels, and sensor data to make the predictions within the geographic area corresponding to where the spill occurred and where the resulting contamination may spread.

Based on those predictions, the system can generate assessments regarding the contamination and issue notifications and/or warnings based on those assessments to one or more entities that may be affected by the contamination. For example, if there are contaminants which are hazardous to consume passing downriver, the system can calculate the amount and/or concentration of those contaminants at different points downriver, at different times. The system can then generate an assessment of the situation. If the assessment indicates no immediate or foreseeable problems, the system can continue normal monitoring of the region. However, if the assessment indicates that there could be a hazardous situation (e.g., by detecting an excessive number of anomalies), the system can issue a warning, notification, and/or other communication to a city, or to citizen scientist organizations, to encourage specific testing protocols and to provide expected timelines for arrival of the contaminants. In addition, the individual entities, can be provided with access to an online or mobile application test result/observation-entry form that, when filled in, can become a part of the event record informing the Al engine of amplitude and/or timing errors. That information can also be used as inputs to support machine learning (ML) routines that refine the model making downstream forecasts. Collected data can also be entered into a reference library to support automated refinement of the pattern recognition function. Further, contaminant load and arrival times can be updated to refine downstream locations to inform their testing protocol and timing plans.

The atmospheric, soil, and/or hydrological models can be machine learning models, where the models are trained using known data detailing how chemicals with known properties (molecular weights, dissipation curve, photosensitivity, etc.) have performed in similar circumstances. When sensor data, or testing data, is collected, that data can be provided to retrain the atmospheric, soil, and/or hydrological models. In this manner, the predictions made by the machine learning models can constantly be improving, allowing for more accurate forecasts to be made regarding what contaminants will be present under various conditions.

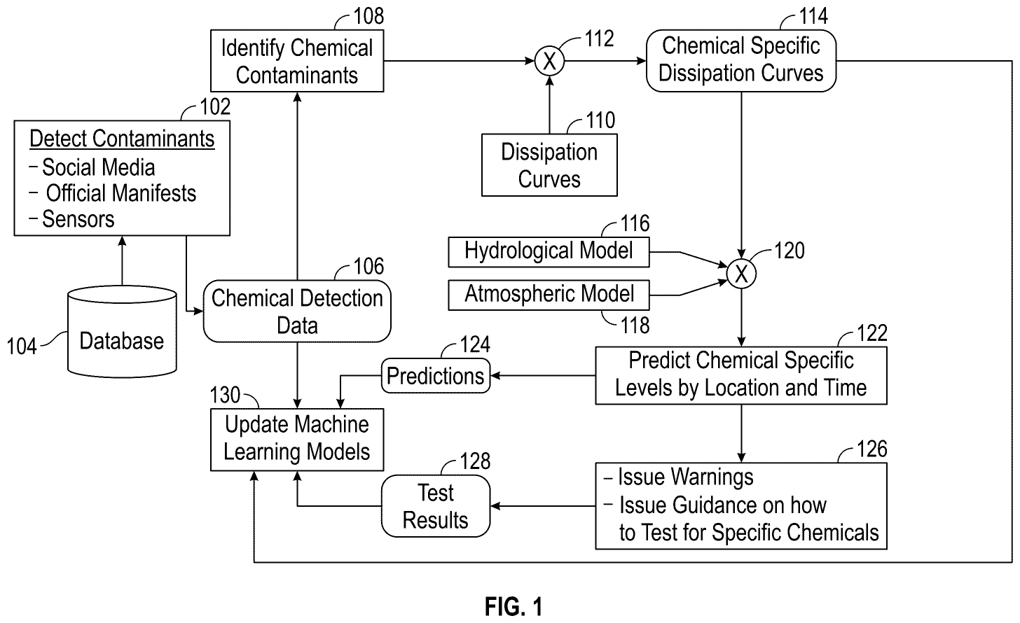

FIG. 1 illustrates an example system embodiment. In this example, the system attempts to detect contaminants 102 from various sources, including social media, official manifest, sensors, and/or a database 104 containing historical records or other stored data. The system aggregates these different pieces of data together, resulting in chemical detection data 106, which is used to identify potentially present chemical contaminants 108. This identification can be based on a comparison and/or a correlation of the chemical detection data 106 to behaviors of known chemicals.

Once the predicted chemical contaminants are identified 108, the system selects 112, for each chemical contaminant, a dissipation curve 110 from a database of known dissipation curves, resulting in chemical specific dissipation curves. That is, if the system had identified 108 chemicals A, B, and C, the system could select dissipation curves for A, B, and C. The chemical dissipation curves 114 identify how long it takes for a chemical to dissipate and/or disperse to the point where it becomes immeasurable and/or insignificant.

The chemical dissipation curves 114 are then applied 120 to hydrological 116 and/or atmospheric 118 models which predict how the chemical will travel over time. Preferably, the models 116, 118 are combined into a single model, though in some configurations the models 116, 118 may be separate and the results of the models can be combined using weighting or other means. Combined with the chemical dissipation curves 114, the output of the models 116, 118 is a prediction of chemical specific levels by location and time 122. The system can, in some configurations, generate the predictions in parallel for each identified chemical contaminant, such that the dissipation and/or dispersion of all the chemicals is processed at the same time. In other configurations, the order of computation can vary, such that some chemicals are analyzed before other chemicals. For example, if some chemicals are more dangerous, the system may prioritize the predicted dispersion and dissipation of those chemicals before analyzing others. Based on the predictions by location and time 122, the system can issue warnings 126 regarding the likelihood that certain chemicals are present, and/or guidance on how to test for those chemicals. Those warnings 126 can, for example, go to cities, counties, non-profit organizations, news organizations, or other entities that may be affected by the contaminants. Those entities may then conduct tests to see if there is any danger, creating test results 128.

The chemical detection data 106, the chemical specific dissipation curves, the predictions 124, the accompanying test results 128 can then be used to update 130 the various machine-learning models within the system. Non-limiting examples of the machine learning models used by the system can include: a model to identify potentially present chemical contaminants 108, the hydrological model 116, the atmospheric model 118, and the prediction of chemical specific levels by location and time 122. In some configurations one or more of those models may be combined. Over time, and with additional data confirming or rejecting the predictions 124, the system can make more efficient and accurate predictions.

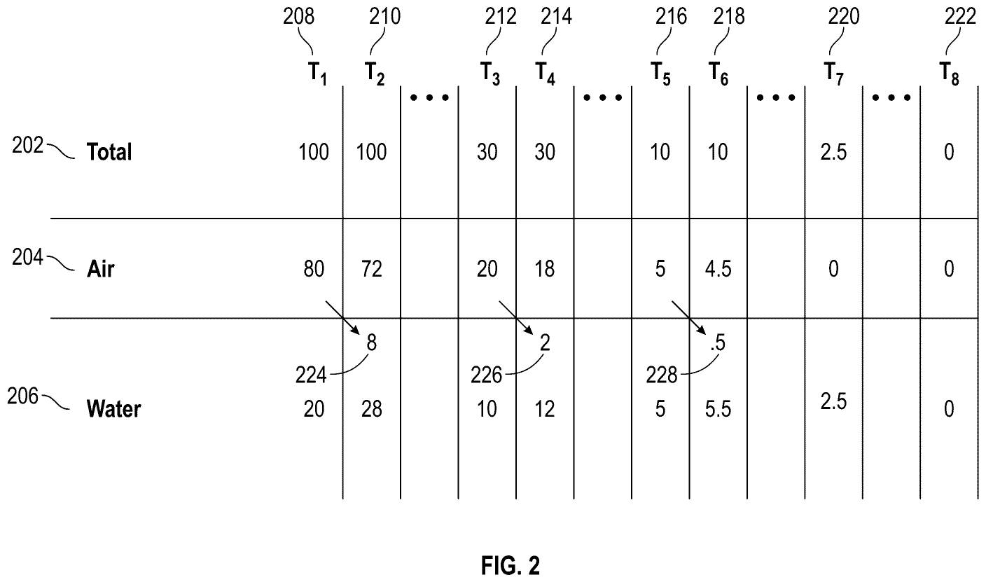

FIG. 2 illustrates an example of a single contaminant dissipating/dispersing over time at a single location. For each time period 208-222, the system calculates a total 202 amount of the contaminant at that time over a combination of air 204 and water 206. Beginning at T₁ 208, the total 202 is 100 (this example is unit agnostic—it could be 100 kilograms, 100 pounds, etc.), with a split of 80 air 204 and 20 water 206. At T₂ 210, eight units of air 224 are transferred to the water while maintaining a total of 100, resulting 72 units of the contaminant in the air and 28 units in the water. At T₃ 212, the total 202 amount of the contaminant has been reduced to thirty, with twenty units in the air and ten units in the water. At T₄ 214, two units of air 226 are transferred to the water while maintaining a total of thirty, resulting in eighteen units in the air and twelve units in the water. Likewise, at T₅ 216 the total 202 amount of the contaminant has been reduced to ten, with five units in the air and five units in the water. At T₆ 218, half a unit 228 of air is transferred to the water while maintaining a total of ten, resulting in four and a half units in the air and five and a half units in the water. At T₇ 220, the total amount of contaminant is two and a half, with no contaminant in the air and two and a half units in the water. Finally, at T₈ 222, the contamination is dissipated/dispersed to the point that no contamination can be detected.

While in example illustrated in FIG. 2 the contaminant was not transferring from the water 206 to the air 204, in other instances (e.g., with other chemicals) such a transfer can be possible. Likewise, the illustrated example only shows two zones—air 204 and water 206, whereas in other examples soil contamination may also be considered.

While the illustrated example of FIG. 2 illustrates a single contaminant dissipating/dispersing over time at a single location, the system disclosed herein can produce similar calculations for all identified or predicted contaminants over multiple locations (e.g., all downwind or downriver locations). As stated above, such calculations can be executed in a parallel fashion or may be executed in a prioritized fashion (e.g., where certain chemicals are analyzed based on a level of danger or lethality).

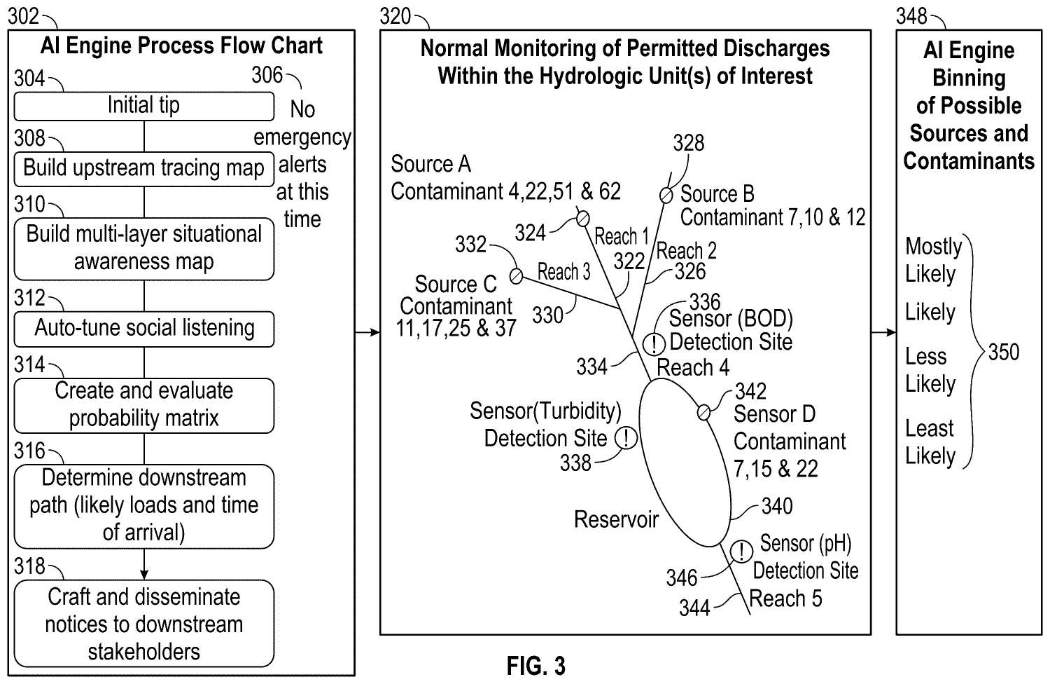

FIG. 3 illustrates an example of normal monitoring of permitted discharges. As illustrated, the AI engine performs steps including (but not limited to): Receiving an initial tip 304; building an upstream tracing map 308 (identifying hydrological systems; in some configurations this can be downstream as well); building a multi-layer situational awareness map 310 (e.g., overlaying the tracing map with sensor data, known contaminant locations, types of contaminants, etc.); auto-tuning social listening 312 (e.g., tuning to specific works in social media); creating and evaluating a probability matrix 314; determining downstream path 316 (e.g., likely loads and time of arrival for a given contamination, and specific types of contaminants within that contamination); and crafting and disseminating notices to downstream stakeholders 318 (e.g., identifying contact information for a downstream locality or jurisdiction, and sending them information regarding the likely spread of contaminants). In this example, the AI engine 302 has detected no emergency alerts 306 at this time, and continues to monitor different locations (e.g., Sources A 524 (covering reach 1 322), B 328 (covering reach 2 326), C 332 (covering reach 3 330), and D 342 (covering reservoir 340)).

Within the hydrological system, there can be specific sensors. As illustrated, this hydrologic system has a sensor detection site 336 monitoring reach 4 334, with that sensor being a Biochemical Oxygen Demand (BOD) sensor. At a point on the illustrated reservoir 340, a sensor detection site 338 detects turbidity. At yet another sensor detection site 346 on reach 5 344, a sensor detects pH of the water. Such sensors are exemplary only, and other types of sensors can be deployed to monitor the quality of the water. In some configurations, the hydrological model illustrated can be generated during passive monitoring, whereas in other configurations the hydrological model may be generated upon receiving tips or other information that there could be contamination within a geographic region.

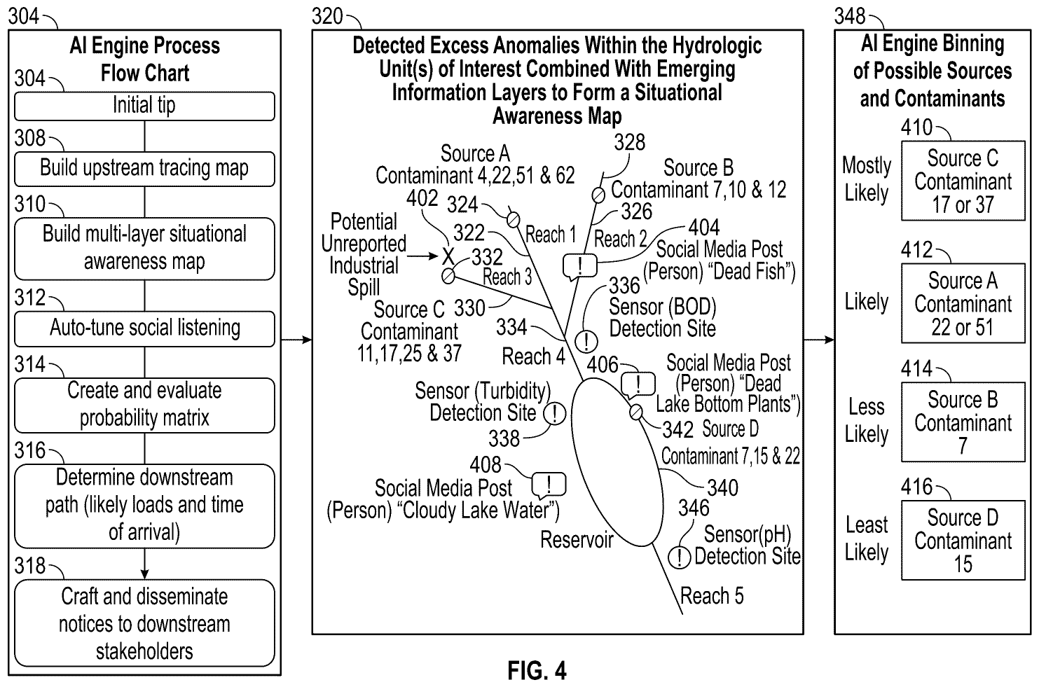

Once potential contamination is detected, the AI engine can generate a multi-layer situational awareness map (see FIG. 4). The AI engine can also auto-tune social listening (e.g., modify tags, locations, or words used to focus acquisition of additional data regarding the potential contamination). With the additional data, the system can create and evaluate a probability matrix for the locations and/or types of contaminants that are present within the region represented by the hydrological model, and determine the downstream path of any contaminants (i.e., likely loads and time of arrival). For example, the AI engine can identify which sources and/or contaminants will be present and “bin” (organize or sort) those sources and contaminants 348 according to predetermined categories (i.e., “bins”) 350, such as (but not limited to) “most likely”, “likely”, “less likely”, and “least likely.” Based on that information the system can make assessments and, if necessary, based on those assessments, craft and disseminate notices to downstream stakeholders.

FIG. 4 illustrates an example of a multi-layer situational awareness map once potential contaminants have been detected. As illustrated, various social media posts have been recorded which indicate that there might be a problem (e.g., “Cloudy lake water” 408, “Dead fish” 404, “Dead Lake Bottom Plants” 406). The system can record those social media posts and place them by location (based on the locations associated with the posts) within the hydrological model. Using the social media posts, known locations of sensors with the accompanying sensor data, and known locations of contaminants, the system can determine a likely location for a potential unreported industrial spill 402 or other source of the new contamination. The AI engine can also “bin” the possible sources with accompanying contaminants, meaning that the possible locations where the new spill occurred are ranked with likely contaminants located in those locations. If there are multiple possible contaminants which are likely for a given location, all those possible contaminants can be listed within the bin for a given location. In this example, the AI engine has determined that Source C 332 is most likely to be the source of contaminants 17 or 37 (410); Source A is likely to be a source of contamination for contaminants 22 or 51 (412); Source B is less likely to be a source for contaminant 7 (414); and Source D is least likely to be a source of contaminant 15 (416).

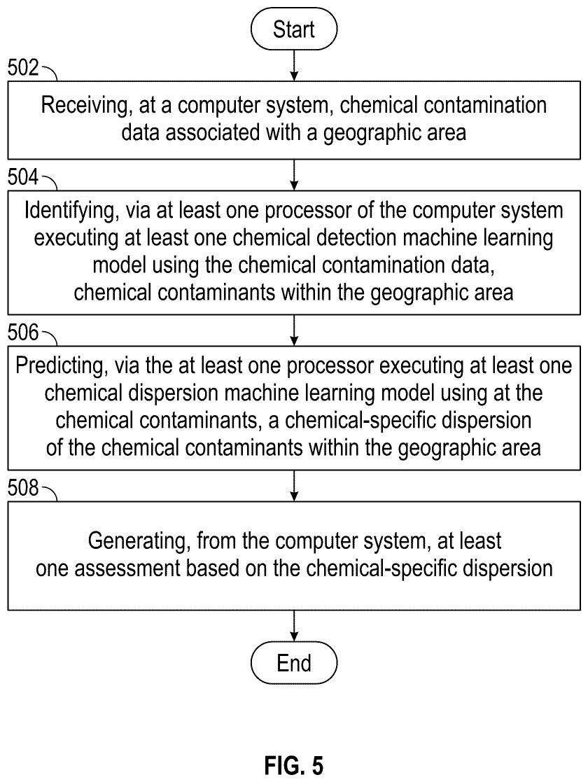

FIG. 5 illustrates an example method embodiment. In this example, the method includes: receiving, at a computer system, chemical contamination data associated with a geographic area (502); identifying, via at least one processor of the computer system executing at least one chemical detection machine learning model using the chemical contamination data, chemical contaminants within the geographic area (504); predicting, via the at least one processor executing at least one chemical dispersion machine learning model using at the chemical contaminants, a chemical-specific dispersion of the chemical contaminants within the geographic area (506); and generating, from the computer system, at least one assessment based on the chemical-specific dispersion (508).

In some configurations, the illustrated method can further include: generating a notification (such as a warning, instructions to test within a window of time, or “all clear”) based on the at least one assessment.

In some configurations, the predicting of the chemical-specific dispersion can further include: generating, via the at least one processor, a chemical-specific dissipation curve for each chemical contaminant in the chemical contaminants, resulting in chemical-specific dissipation curves, where inputs to the at least one chemical dispersion machine learning model comprise: the chemical-specific dissipation curves; and locations of the chemical contaminants within the geographic area. In such configurations, the generating of the chemical-specific dissipation curve for each chemical contaminant in the chemical contaminants further comprises: identifying, within a database, previously developed chemically agnostic dissipation curves; and selecting, for each chemical contaminant in the chemical contaminants, a previously developed chemically agnostic dissipation curve from within the previously developed chemically agnostic dissipation curves, resulting in the chemical-specific dissipation curves.

In some configurations the inputs to the chemical dispersion machine learning model can further include: at least one hydrological model associated with the geographic area; and at least one atmospheric model associated with the geographic area. In such configurations the inputs to the chemical dispersion machine learning model can further include: a weather forecast.

In some configurations the chemical contamination data can be received from scraping social media data. In such configurations, the scraping of the social media data can further include correlating keywords detected within the social media data to effects of chemical contaminants.

In some configurations the chemical contamination data can include at least one of official manifests, sensor data, and social media data.

In some configurations the illustrated method can further include: receiving, at the computer system in response to the at least one warning, test results from the at least one entity; updating the chemical detection machine learning model based on the test results; and updating the chemical dispersion machine learning model based on the test results.

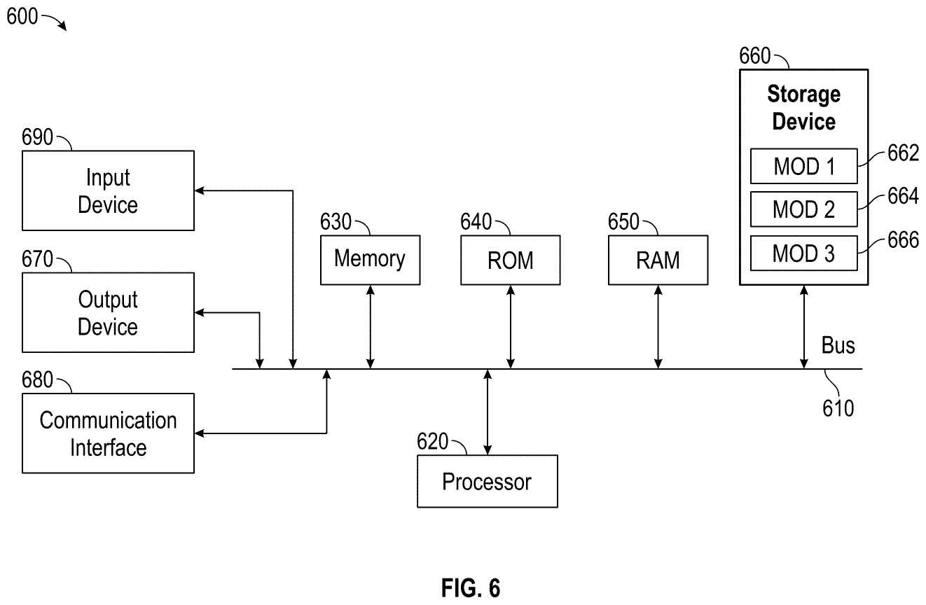

With reference to FIG. 6, an exemplary system includes a general-purpose computing device 600, including a processing unit (CPU or processor) 620 and a system bus 610 that couples various system components including the system memory 630 such as read-only memory (ROM) 640 and random-access memory (RAM) 650 to the processor 620. The system 600 can include a cache of high-speed memory connected directly with, in close proximity to, or integrated as part of the processor 620. The system 600 copies data from the memory 630 and/or the storage device 660 to the cache for quick access by the processor 620. In this way, the cache provides a performance boost that avoids processor 620 delays while waiting for data. These and other modules can control or be configured to control the processor 620 to perform various actions. Other system memory 630 may be available for use as well. The memory 630 can include multiple different types of memory with different performance characteristics. It can be appreciated that the disclosure may operate on a computing device 600 with more than one processor 620 or on a group or cluster of computing devices networked together to provide greater processing capability. The processor 620 can include any general-purpose processor and a hardware module or software module, such as module 1 662, module 2 664, and module 3 666 stored in storage device 660, configured to control the processor 620 as well as a special-purpose processor where software instructions are incorporated into the actual processor design. The processor 620 may essentially be a completely self-contained computing system, containing multiple cores or processors, a bus, memory controller, cache, etc. A multi-core processor may be symmetric or asymmetric.

The system bus 610 may be any of several types of bus structures including a memory bus or memory controller, a peripheral bus, and a local bus using any of a variety of bus architectures. A basic input/output (BIOS) stored in ROM 640 or the like, may provide the basic routine that helps to transfer information between elements within the computing device 600, such as during start-up. The computing device 600 further includes storage devices 660 such as a hard disk drive, a magnetic disk drive, an optical disk drive, tape drive or the like. The storage device 660 can include software modules 662, 664, 666 for controlling the processor 620. Other hardware or software modules are contemplated. The storage device 660 is connected to the system bus 610 by a drive interface. The drives and the associated computer-readable storage media provide nonvolatile storage of computer-readable instructions, data structures, program modules and other data for the computing device 600. In one aspect, a hardware module that performs a particular function includes the software component stored in a tangible computer-readable storage medium in connection with the necessary hardware components, such as the processor 620, bus 610, display 670, and so forth, to carry out the function. In another aspect, the system can use a processor and computer-readable storage medium to store instructions which, when executed by a processor (e.g., one or more processors), cause the processor to perform a method or other specific actions. The basic components and appropriate variations are contemplated depending on the type of device, such as whether the device 600 is a small, handheld computing device, a desktop computer, or a computer server.

Although the exemplary embodiment described herein employs the hard disk 660, other types of computer-readable media which can store data that are accessible by a computer, such as magnetic cassettes, flash memory cards, digital versatile disks, cartridges, random access memories (RAMs) 650, and read-only memory (ROM) 640, may also be used in the exemplary operating environment. Tangible computer-readable storage media, computer-readable storage devices, or computer-readable memory devices, expressly exclude media such as transitory waves, energy, carrier signals, electromagnetic waves, and signals per se.

To enable user interaction with the computing device 600, an input device 690 represents any number of input mechanisms, such as a microphone for speech, a touch-sensitive screen for gesture or graphical input, keyboard, mouse, motion input, speech and so forth. An output device 670 can also be one or more of a number of output mechanisms known to those of skill in the art. In some instances, multimodal systems enable a user to provide multiple types of input to communicate with the computing device 600. The communications interface 680 generally governs and manages the user input and system output. There is no restriction on operating on any particular hardware arrangement and therefore the basic features here may easily be substituted for improved hardware or firmware arrangements as they are developed.

The technology discussed herein refers to computer-based systems and actions taken by, and information sent to and from, computer-based systems. One of ordinary skill in the art will recognize that the inherent flexibility of computer-based systems allows for a great variety of possible configurations, combinations, and divisions of tasks and functionality between and among components. For instance, processes discussed herein can be implemented using a single computing device or multiple computing devices working in combination. Databases, memory, instructions, and applications can be implemented on a single system or distributed across multiple systems. Distributed components can operate sequentially or in parallel.

Use of language such as “at least one of X, Y, and Z,” “at least one of X, Y, or Z,” “at least one or more of X, Y, and Z,” “at least one or more of X, Y, or Z,” “at least one or more of X, Y, and/or Z,” or “at least one of X, Y, and/or Z,” are intended to be inclusive of both a single item (e.g., just X, or just Y, or just Z) and multiple items (e.g., {X and Y}, {X and Z}, {Y and Z}, or {X, Y, and Z}). The phrase “at least one of” and similar phrases are not intended to convey a requirement that each possible item must be present, although each possible item may be present.

The various embodiments described above are provided by way of illustration only and should not be construed to limit the scope of the disclosure. Various modifications and changes may be made to the principles described herein without following the example embodiments and applications illustrated and described herein, and without departing from the spirit and scope of the disclosure. For example, unless otherwise explicitly indicated, the steps of a process or method may be performed in an order other than the example embodiments discussed above. Likewise, unless otherwise indicated, various components may be omitted, substituted, or arranged in a configuration other than the example embodiments discussed above.

Further aspects of the present disclosure are provided by the subject matter of the following clauses.

A method comprising: receiving, at a computer system, chemical contamination data associated with a geographic area; identifying, via at least one processor of the computer system executing at least one chemical detection machine learning model using the chemical contamination data, chemical contaminants within the geographic area; predicting, via the at least one processor executing at least one chemical dispersion machine learning model using at the chemical contaminants, a chemical-specific dispersion of the chemical contaminants within the geographic area; and generating, from the computer system, at least one assessment based on the chemical-specific dispersion.

The method of any preceding clause, further comprising: generating a notification based on the at least one assessment.

The method of any preceding clause, wherein the predicting of the chemical-specific dispersion further comprises: generating, via the at least one processor, a chemical-specific dissipation curve for each chemical contaminant in the chemical contaminants, resulting in chemical-specific dissipation curves, wherein inputs to the at least one chemical dispersion machine learning model comprise: the chemical-specific dissipation curves; and locations of the chemical contaminants within the geographic area.

The method of any preceding clause, wherein the generating of the chemical-specific dissipation curve for each chemical contaminant in the chemical contaminants further comprises: identifying, within a database, previously developed chemically agnostic dissipation curves; and selecting, for each chemical contaminant in the chemical contaminants, a previously developed chemically agnostic dissipation curve from within the previously developed chemically agnostic dissipation curves, resulting in the chemical-specific dissipation curves.

The method of any preceding clause, wherein the inputs to the chemical dispersion machine learning model further comprise: at least one hydrological model associated with the geographic area; and at least one atmospheric model associated with the geographic area.

The method of any preceding clause, wherein the inputs to the chemical dispersion machine learning model further comprise: a weather forecast.

The method of any preceding clause, wherein the chemical contamination data is received from scraping social media data.

The method of any preceding clause, wherein the scraping of the social media data further comprises correlating keywords detected within the social media data to effects of chemical contaminants.

The method of any preceding clause, wherein the chemical contamination data comprises at least one of official manifests, sensor data, and social media data.

The method of any preceding clause, further comprising: receiving, at the computer system in response to the at least one warning, test results from at least one entity; updating the chemical detection machine learning model based on the test results; and updating the chemical dispersion machine learning model based on the test results.

A system, comprising: at least one processor; and a non-transitory computer-readable storage medium having instructions stored which, when executed by the at least one processor, cause the at least one processor to perform operations comprising: receiving chemical contamination data associated with a geographic area; identifying, by executing at least one chemical detection machine learning model using the chemical contamination data, chemical contaminants within the geographic area; predicting, by executing at least one chemical dispersion machine learning model using at the chemical contaminants, a chemical-specific dispersion of the chemical contaminants within the geographic area; and issuing at least one warning to at least one entity based on the chemical-specific dispersion.

The system of any preceding clause, wherein the predicting of the chemical-specific dispersion further comprises: generating a chemical-specific dissipation curve for each chemical contaminant in the chemical contaminants, resulting in chemical-specific dissipation curves, wherein inputs to the at least one chemical dispersion machine learning model comprise: the chemical-specific dissipation curves; and locations of the chemical contaminants within the geographic area.

The system of any preceding clause, wherein the generating of the chemical-specific dissipation curve for each chemical contaminant in the chemical contaminants further comprises: identifying, within a database, previously developed chemically agnostic dissipation curves; and selecting, for each chemical contaminant in the chemical contaminants, a previously developed chemically agnostic dissipation curve from within the previously developed chemically agnostic dissipation curves, resulting in the chemical-specific dissipation curves.

The system of any preceding clause, wherein the inputs to the chemical dispersion machine learning model further comprise: at least one hydrological model associated with the geographic area; and at least one atmospheric model associated with the geographic area.

The system of any preceding clause, wherein the inputs to the chemical dispersion machine learning model further comprise: a weather forecast.

The system of any preceding clause, wherein the chemical contamination data is received from scraping social media data.

The system of any preceding clause, wherein the scraping of the social media data further comprises correlating keywords detected within the social media data to effects of chemical contaminants.

The system of any preceding clause, wherein the chemical contamination data comprises at least one of official manifests, sensor data, and social media data.

The system of any preceding clause, the non-transitory computer-readable storage medium having additional instructions stored which, when executed by the at least one processor, cause the at least one processor to perform operations comprising: receiving, in response to the at least one warning, test results from at least one entity; updating the chemical detection machine learning model based on the test results; and updating the chemical dispersion machine learning model based on the test results.

A non-transitory computer-readable storage medium having instructions stored which, when executed by at least one processor, cause the at least one processor to perform operations comprising: receiving chemical contamination data associated with a geographic area; identifying, by executing at least one chemical detection machine learning model using the chemical contamination data, chemical contaminants within the geographic area; predicting, by executing at least one chemical dispersion machine learning model using at the chemical contaminants, a chemical-specific dispersion of the chemical contaminants within the geographic area; and issuing at least one warning to at least one entity based on the chemical-specific dispersion.

The non-transitory computer-readable storage medium of any preceding clause, wherein the predicting of the chemical-specific dispersion further comprises: generating a chemical-specific dissipation curve for each chemical contaminant in the chemical contaminants, resulting in chemical-specific dissipation curves, wherein inputs to the at least one chemical dispersion machine learning model comprise: the chemical-specific dissipation curves; and locations of the chemical contaminants within the geographic area.

We claim:

1. A method comprising:
receiving, at a computer system, chemical contamination data associated with a geographic area;
identifying, via at least one processor of the computer system executing at least one chemical detection machine learning model using the chemical contamination data, chemical contaminants within the geographic area;
predicting, via the at least one processor executing at least one chemical dispersion machine learning model using at the chemical contaminants, a chemical-specific dispersion of the chemical contaminants within the geographic area; and
generating, from the computer system, at least one assessment based on the chemical-specific dispersion.

2. The method of claim 1, further comprising:
generating a notification based on the at least one assessment.

3. The method of claim 1, wherein the predicting of the chemical-specific dispersion further comprises:
generating, via the at least one processor, a chemical-specific dissipation curve for each chemical contaminant in the chemical contaminants, resulting in chemical-specific dissipation curves,
wherein inputs to the at least one chemical dispersion machine learning model comprise:
the chemical-specific dissipation curves; and
locations of the chemical contaminants within the geographic area.

4. The method of claim 3, wherein the generating of the chemical-specific dissipation curve for each chemical contaminant in the chemical contaminants further comprises:
identifying, within a database, previously developed chemically agnostic dissipation curves; and
selecting, for each chemical contaminant in the chemical contaminants, a previously developed chemically agnostic dissipation curve from within the previously developed chemically agnostic dissipation curves, resulting in the chemical-specific dissipation curves.

5. The method of claim 1, wherein inputs to the chemical dispersion machine learning model comprise:
at least one hydrological model associated with the geographic area; and
at least one atmospheric model associated with the geographic area.

6. The method of claim 5, wherein the inputs to the chemical dispersion machine learning model further comprise:
a weather forecast.

7. The method of claim 1, wherein the chemical contamination data is received from scraping social media data.

8. The method of claim 7, wherein the scraping of the social media data further comprises correlating keywords detected within the social media data to effects of chemical contaminants.

9. The method of claim 1, wherein the chemical contamination data comprises at least one of official manifests, sensor data, and social media data.

10. The method of claim 1, further comprising:
receiving, at the computer system in response to the at least one warning, test results from at least one entity;
updating the chemical detection machine learning model based on the test results; and
updating the chemical dispersion machine learning model based on the test results.

11. A system, comprising:
at least one processor; and
a non-transitory computer-readable storage medium having instructions stored which, when executed by the at least one processor, cause the at least one processor to perform operations comprising:
receiving chemical contamination data associated with a geographic area;
identifying, by executing at least one chemical detection machine learning model using the chemical contamination data, chemical contaminants within the geographic area;
predicting, by executing at least one chemical dispersion machine learning model using at the chemical contaminants, a chemical-specific dispersion of the chemical contaminants within the geographic area; and
generating at least one assessment based on the chemical-specific dispersion.

12. The system of claim 11, wherein the predicting of the chemical-specific dispersion further comprises:
generating a chemical-specific dissipation curve for each chemical contaminant in the chemical contaminants, resulting in chemical-specific dissipation curves, wherein inputs to the at least one chemical dispersion machine learning model comprise:
the chemical-specific dissipation curves; and
locations of the chemical contaminants within the geographic area.

13. The system of claim 12, wherein the generating of the chemical-specific dissipation curve for each chemical contaminant in the chemical contaminants further comprises:
identifying, within a database, previously developed chemically agnostic dissipation curves; and
selecting, for each chemical contaminant in the chemical contaminants, a previously developed chemically agnostic dissipation curve from within the previously developed chemically agnostic dissipation curves, resulting in the chemical-specific dissipation curves.

14. The system of claim 10, wherein inputs to the chemical dispersion machine learning model further comprise:
at least one hydrological model associated with the geographic area; and
at least one atmospheric model associated with the geographic area.

15. The system of claim 13, wherein the inputs to the chemical dispersion machine learning model further comprise:
a weather forecast.

16. The system of claim 10, wherein the chemical contamination data is received from scraping social media data.

17. The system of claim 16, wherein the scraping of the social media data further comprises correlating keywords detected within the social media data to effects of chemical contaminants.

18. The system of claim 10, wherein the chemical contamination data comprises at least one of official manifests, sensor data, and social media data.

19. The system of claim 11, having additional instructions stored which, when executed by the at least one processor, cause the at least one processor to perform operations comprising:
receiving, in response to the at least one warning, test results from at least one entity;
updating the chemical detection machine learning model based on the test results; and
updating the chemical dispersion machine learning model based on the test results.

20. A non-transitory computer-readable storage medium having instructions stored which, when executed by at least one processor, cause the at least one processor to perform operations comprising:
receiving chemical contamination data associated with a geographic area;
identifying, by executing at least one chemical detection machine learning model using the chemical contamination data, chemical contaminants within the geographic area;
predicting, by executing at least one chemical dispersion machine learning model using at the chemical contaminants, a chemical-specific dispersion of the chemical contaminants within the geographic area; and
issuing at least one warning to at least one entity based on the chemical-specific dispersion.
